(19) United States
(12) Patent Application Publication
(10) Pub. No.: US 2026/0272313 A1
(43) Pub. Date: Sep. 17, 2026

(54) BLOOD PRESSURE MEASUREMENT DEVICE, BIOLOGICAL INFORMATION PROCESSING SYSTEM, INFORMATION PROCESSING TERMINAL, AND STORAGE MEDIUM

(71) Applicant: OMRON HEALTHCARE Co., Ltd., Muko-shi (JP)

(72) Inventors: Shingo YAMASHITA, Kyoto (JP); Masaki YAMADA, Kyoto (JP)

(73) Assignee: OMRON HEALTHCARE Co., Ltd., Muko-shi (JP)

(21) Appl. No.: 19/531,864

(22) Filed: Feb. 6, 2026

(30) Foreign Application Priority Data
Mar. 11, 2025 (JP) 2025-038637

(51) Int. Cl. A61B 5/021; A61B 5/00; A61B 5/0205; A61B 5/024; A61B 5/11

(52) U.S. Cl. CPC A61B 5/02108; A61B 5/0205; A61B 5/024; A61B 5/11; A61B 5/742

(57) ABSTRACT

A blood pressure measurement device measures blood pressure and includes a pulse wave acquisition unit, a display unit, and a control unit. During pulse wave acquisition, if one or more indices meet an arrhythmia detection condition, the control unit determines if arrhythmia is present. If the condition is met and arrhythmia is detected, the display shows that arrhythmia was present during the acquisition. If the condition is not met, the display indicates that arrhythmia determination was not performed. This assesses the feasibility of arrhythmia detection based on the indices and displays the result, promoting more reliable blood pressure readings.

### CROSS-REFERENCE TO RELATED APPLICATIONS

The disclosure of Japanese Patent Application No.JP 2025-038637, filed on March 11, 2025 including the specification, drawings and abstract is incorporated herein by reference in its entirety.

### TECHNICAL FIELD

The present invention relates to a blood pressure measurement device, a biological information processing system, an information processing terminal, and a program.

### BACKGROUND ART

In recent years, it has become widespread to perform health management by measuring information regarding the body and health of an individual such as a blood pressure value with a measuring instrument and recording and analyzing the measurement result. In particular, since arrhythmia such as atrial fibrillation (AF) may lead to a brain/cardiovascular disease, it is desirable that the user himself/herself can easily recognize such an abnormality.

For example, Patent Document 1 discloses a biological information system that presents periods during which arrhythmia has occurred within a predetermined period to a user on the basis of pulse wave information or the like acquired by a wearable device. Furthermore, Patent Document 1 proposes a technology in which reliability of the periods during which arrhythmia has occurred is obtained on the basis of information such as body motion during the periods that have been determined as the periods during which arrhythmia has occurred, and, for the periods with low reliability, the determination that arrhythmia has occurred is canceled, that is, the periods with low reliability are not presented as the period during which arrhythmia has occurred. This makes it possible to prevent a determination result with low reliability from being presented to the user.

### PRIOR ART DOCUMENT

### Patent Document

Patent Document 1: JP 2017-042386 A

### SUMMARY

However, in the technology as described in Patent Document 1, a case where the presentation to the user has been canceled due to the low reliability for the period during which arrhythmia has occurred corresponds to, from the user’s viewpoint, the information that arrhythmia has occurred being not presented. That is, the user cannot distinguish whether “no arrhythmia has been detected” or “the detection of arrhythmia has been canceled”. Therefore, even in a case where the detection of arrhythmia has been actually canceled (the determination as to whether or not arrhythmia has occurred is suspended), the user may erroneously recognize that “arrhythmia has not occurred in the period”.

In view of the above circumstances, an object of the present invention is to provide a technology for enabling, in a case where the determination of the presence or absence of arrhythmia has not been performed in a blood pressure measurement device with an arrhythmia detection function, a user to grasp a fact that the determination of the presence or absence of arrhythmia has not been performed.

In order to solve the above problem, the present invention adopts the following configuration. The configuration is

a blood pressure measurement device that measures blood pressure of a living body, the blood pressure measurement device including:

a pulse wave acquisition unit that acquires pulse wave information;

a display unit including an area capable of displaying at least the blood pressure measured; and

a control unit that performs control of the blood pressure measurement device, wherein

the control unit

performs determination of presence or absence of arrhythmia during acquisition of the pulse wave information in a case where one or more indexes satisfy an arrhythmia detection condition, the one or more indexes being related to determination of possibility or impossibility of detection of arrhythmia in the living body at a time of the acquisition of the pulse wave information,

causes, in a case where the one or more indexes have satisfied the arrhythmia detection condition, and where it has been determined that arrhythmia has been present during the acquisition of the pulse wave information, the display unit to display information of a fact that arrhythmia has been present during the acquisition of the pulse wave information, and

causes, in a case where the one or more indexes have not satisfied the arrhythmia detection condition, the display unit to display information of a fact that determination of presence or absence of arrhythmia during the acquisition of the pulse wave information has not been performed.

Note that “arrhythmia” used herein includes atrial fibrillation, atrial flutter, premature contraction, and the like. According to such a configuration, in a case where the arrhythmia has been detected, the fact of that is presented to the user, and in a case where the condition for detecting arrhythmia has not been satisfied and the determination of the presence or absence of arrhythmia has been suspended (the arrhythmia detection processing has not been performed), the information of that fact is presented to the user. That is, the information to be presented to the user can be made different between a case where the determination of the presence or absence of arrhythmia has been performed and then it has been determined that arrhythmia has been absent and a case where the determination of the presence or absence of arrhythmia has not been performed in the first place. Therefore, it is possible to prevent the user from erroneously recognizing that arrhythmia has been absent (eventually leading to a false negative determination) even though the determination of the presence or absence of arrhythmia has merely not actually been executed.

Furthermore, the one or more indexes may include a body motion level during the acquisition of the pulse wave information of the living body, and

the control unit may determine whether or not the arrhythmia detection condition is satisfied using information related to body motion of the living body at the time of the acquisition of the pulse wave information.

Furthermore, the one or more indexes may include a pulse rate level during the acquisition of the pulse wave information of the living body, and

the control unit may determine whether or not the arrhythmia detection condition is satisfied using information on a pulse rate of the living body at the time of the acquisition of the pulse wave information.

Furthermore, in a case where the body motion level during the acquisition of the pulse wave information has been equal to or higher than a threshold value and the one or more indexes have not satisfied the arrhythmia detection condition, the control unit may display, at positions close to each other on the display unit, information of a fact that the determination of presence or absence of arrhythmia has not been performed and information of a fact that the body motion has been detected.

According to such a configuration, the user can easily visually recognize that the determination of the presence or absence of arrhythmia has not been performed due to the body motion as a cause.

Furthermore, the blood pressure measurement device may further include a storage unit that holds information related to measurement of the blood pressure performed in a past, the information being information related to a date and time of the measurement and information related to presence or absence of arrhythmia during the acquisition of the pulse wave information or information of a fact that determination of presence or absence of arrhythmia has not been performed, and

the control unit

may receive a request to display, on the display unit, the information related to the measurement performed in the past of the blood pressure, the information being held in the storage unit,

may cause, in a case where information of a fact that arrhythmia has been present during the acquisition of the pulse wave information is held as the information related to the measurement of the blood pressure related to the request to display, the display unit to display the information of the fact that arrhythmia has been present during the acquisition of the pulse wave information, and may cause, in a case where information of a fact that determination of presence or absence of arrhythmia has not been performed during the acquisition of the pulse wave information is held, the display unit to display information of the fact that determination of presence or absence of arrhythmia has not been performed during the acquisition of the pulse wave information.

With such a configuration, the user can view the past measurement history, and in a case where the arrhythmia has been detected at the time of the past measurement, the user can confirm that fact. In a case where the determination of the presence or absence of arrhythmia has not been performed at the time of the past measurement, the information on that is displayed. Therefore, it is possible to prevent the user from erroneously recognizing that no arrhythmia has been detected.

Furthermore, the present invention can also be regarded as a biological information processing system as follows. The biological information processing system is

a biological information processing system including: a blood pressure measurement device that measures blood pressure of a living body; and an information processing terminal that includes a display unit and is configured to be communicable with the blood pressure measurement device, wherein

the blood pressure measurement device is configured to acquire biological information of the living body including at least pulse wave information and transmit the biological information to the information processing terminal, and wherein

the information processing terminal is configured to

receive the biological information,

perform determination of presence or absence of arrhythmia during acquisition of the pulse wave information in a case where one or more indexes satisfy an arrhythmia detection condition, the one or more indexes being related to determination of possibility or impossibility of detection of arrhythmia in the living body at a time of the acquisition of the pulse wave information,

display, in a case where the one or more indexes have satisfied the arrhythmia detection condition, and where it has been determined that arrhythmia has been present during the acquisition of the pulse wave information, information of a fact that arrhythmia has been present during the acquisition of the pulse wave information on the display unit, and

display, in a case where the one or more indexes have not satisfied the arrhythmia detection condition, information of a fact that determination of presence or absence of arrhythmia during the acquisition of the pulse wave information has not been performed on the display unit.

Furthermore, the one or more indexes may include a body motion level during the acquisition of the pulse wave information of the living body, and the information processing terminal may determine whether or not the arrhythmia detection condition is satisfied using at least information related to body motion of the living body at the time of the acquisition of the pulse wave information.

Furthermore, the one or more indexes may further include frequency of irregular pulse waves during the acquisition of the pulse wave information of the living body, and the information processing terminal may detect the irregular pulse waves of the living body at the time of the acquisition of the pulse wave information on the basis of the biological information, and may determine whether or not the arrhythmia detection condition is satisfied further using information related to the number of times of detection of the irregular pulse waves.

Furthermore, the present invention can also be regarded as an information processing terminal as follows. The information processing terminal is

an information processing terminal including a communication unit, a display unit, and a control unit, and configured to be communicably connectable to a biological information measurement device that acquires biological information including at least pulse wave information, wherein

the control unit

receives the biological information from the biological information measurement device via the communication unit,

performs determination of presence or absence of arrhythmia during acquisition of the pulse wave information in a case where one or more indexes satisfy an arrhythmia detection condition, the one or more indexes being related to determination of possibility or impossibility of detection of arrhythmia in the living body at a time of the acquisition of the pulse wave information,

causes, in a case where the one or more indexes have satisfied the arrhythmia detection condition, and where it has been determined that arrhythmia has been present during the acquisition of the pulse wave information, the display unit to display information of a fact that arrhythmia has been present during the acquisition of the pulse wave information, and

causes, in a case where the one or more indexes have not satisfied the arrhythmia detection condition, the display unit to display information of a fact that determination of presence or absence of arrhythmia during the acquisition of the pulse wave information has not been performed.

Furthermore, the present invention can also be regarded as a program for causing a computer to function as the above-described information processing terminals, and a computer-readable recording medium in which such a program is non-transiently recorded.

Note that each of configurations and processing described above can be combined with each other to constitute the present invention as long as no technical contradiction occurs.

According to the present invention, in a case where the determination of the presence or absence of arrhythmia has not been performed in a blood pressure measurement device with an arrhythmia detection function, a user can grasp the fact that the determination of the presence or absence of arrhythmia has not been performed.

### BRIEF DESCRIPTION OF THE DRAWINGS

FIG. 1 is a block diagram schematically illustrating a device configuration of a blood pressure measurement device according to a first embodiment;

FIG. 2 is a block diagram illustrating functional modules implemented by a control unit of the blood pressure measurement device according to the first embodiment;

FIG. 3A is a first diagram for explaining an arrhythmia detection condition and a blood pressure calculation condition in the first embodiment;

FIG. 3B is a second diagram for explaining the arrhythmia detection condition and the blood pressure calculation condition in the first embodiment;

FIG. 4A is a first diagram illustrating an example of a measurement result screen displayed on a display unit of the blood pressure measurement device according to the first embodiment;

FIG. 4B is a second diagram illustrating an example of the measurement result screen displayed on the display unit of the blood pressure measurement device according to the first embodiment;

FIG. 4C is a third diagram illustrating an example of the measurement result screen displayed on the display unit of the blood pressure measurement device according to the first embodiment;

FIG. 5A is a fourth diagram illustrating an example of the measurement result screen displayed on the display unit of the blood pressure measurement device according to the first embodiment;

FIG. 5B is a fifth diagram illustrating an example of the measurement result screen displayed on the display unit of the blood pressure measurement device according to the first embodiment;

FIG. 5C is a sixth diagram illustrating an example of the measurement result screen displayed on the display unit of the blood pressure measurement device according to the first embodiment;

FIG. 6 is a flowchart illustrating a flow of blood pressure measurement processing by the blood pressure measurement device according to the first embodiment;

FIG. 7 is a flowchart illustrating a flow of processing when a history viewing mode is executed in the blood pressure measurement device according to the first embodiment;

FIG. 8 is a diagram illustrating a modification of the measurement result screen displayed on the display unit of the blood pressure measurement device according to the first embodiment;

FIG. 9 is a block diagram schematically illustrating a biological information processing system according to a second embodiment;

FIG. 10A is a block diagram illustrating functional modules implemented by a control unit of the blood pressure measurement device according to the second embodiment;

FIG. 10B is a block diagram illustrating functional modules implemented by a control unit of an information processing terminal according to the second embodiment;

FIG. 11A is a first diagram illustrating an example of a result screen displayed on the information processing terminal according to the second embodiment;

FIG. 11B is a second diagram illustrating an example of the result screen displayed on the information processing terminal according to the second embodiment;

FIG. 12 is a flowchart illustrating a flow of processing by the blood pressure measurement device in the biological information processing system according to the second embodiment; and

FIG. 13 is a flowchart illustrating a flow of processing by the information processing terminal in the biological information processing system according to the second embodiment.

### DESCRIPTION OF THE EMBODIMENTS

### First Embodiment

Hereinafter, specific embodiments of the present invention will be described on the basis of the drawings. However, the specific detail of each configuration described in the embodiments described below, the arrangement relationship thereof, and the like are not intended to limit the scope of the present invention only to them unless otherwise specified.

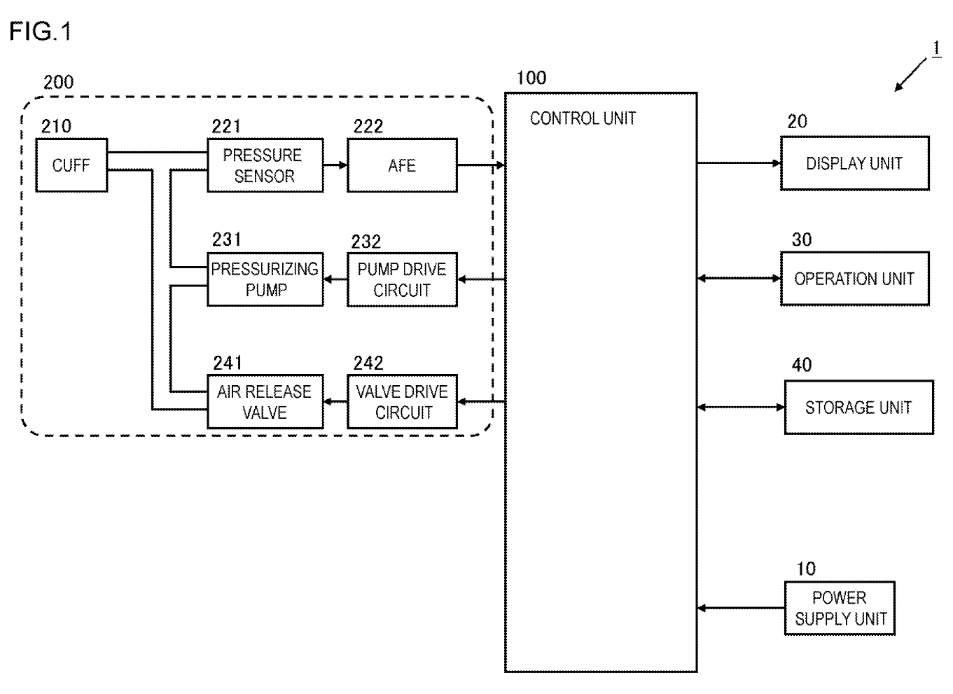

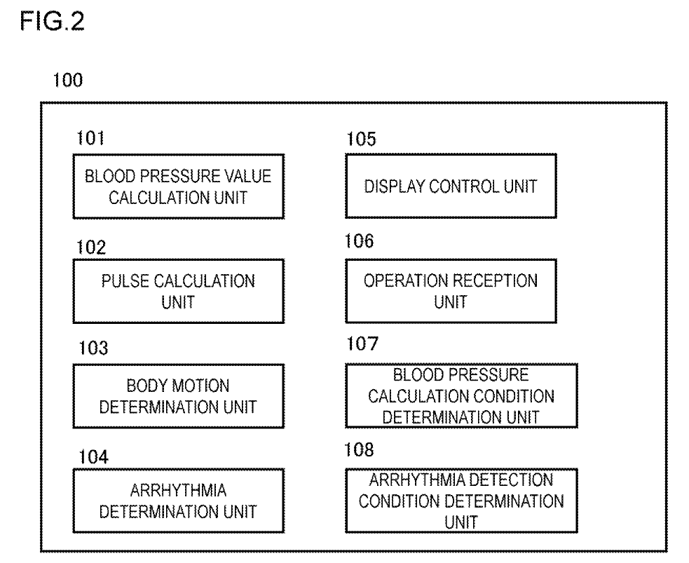

The present invention can be applied to, for example, a blood pressure measurement device 1 as illustrated in FIGS. 1 and 2. FIG. 1 is a block diagram schematically illustrating a device configuration of the blood pressure measurement device 1 in the present embodiment, and FIG. 2 is a block diagram illustrating an example of functional modules implemented by a control unit 100 of the blood pressure measurement device 1.

Although not illustrated, the blood pressure measurement device 1 according to the present embodiment can have an appearance similar to that of a general portable upper-arm sphygmomanometer including a main body housing, a cuff, and an air tube, and as illustrated in FIG. 1, schematically includes components including a power supply unit 10, a display unit 20, an operation unit 30, a storage unit 40, a control unit 100, and a pulse wave information acquisition unit 200.

The power supply unit 10 is a power source of the blood pressure measurement device 1, and may be, for example, a rechargeable secondary battery such as a lithium ion battery, but is not limited thereto, and may be a primary battery, a terminal connectable to an external power supply, or the like.

The display unit 20 is a functional unit for displaying various types of information, and can be, for example, a display device such as a liquid crystal display (LCD). Furthermore, the operation unit 30 is a functional unit that receives a user operation such as turning on/off of a power supply, execution of measurement, and selection/decision of a menu, and can be configured by an operation switch and the like. Note that a touch panel display can also be adopted as a configuration that serves as both the display unit 20 and the operation unit 30.

The storage unit 40 includes a main storage device such as a random access memory (RAM) and a read only memory (ROM), and an auxiliary storage device such as an HDD and a flash memory, and stores various types of information such as an application program, measurement results such as a blood pressure value and a pulse, information on the presence or absence of arrhythmia to be described later, and other acquired biological information. Note that the measurement results of the blood pressure and the like and other acquired biological information may be stored in the storage unit 40 in association with time information such as the measurement (acquisition) time. As the time information, for example, time information clocked by referring to a real time clock (RTC) of the control unit 100 to be described later can be used. Furthermore, the auxiliary storage device may be configured to be detachable from the main body housing.

The control unit 100 is a functional unit that controls the entire blood pressure measurement device 1, and includes, for example, a processor such as a central processing unit (CPU). When receiving a user's operation via the operation unit 30, the control unit 100 controls each component of the blood pressure measurement device 1 to execute various processing such as blood pressure measurement and presentation of various types of information according to a predetermined program. The predetermined program is stored in the storage unit 40 and read therefrom. As the processor, in addition to the CPU, a graphic processing unit (GPU), a field programmable grid array (FPGA), an application specific integrated circuit (ASIC), or the like can be adopted.

Furthermore, the control unit 100 includes functional modules illustrated in FIG. 2, including a blood pressure value calculation unit 101, a pulse calculation unit 102, a body motion determination unit 103, an arrhythmia determination unit 104, a display control unit 105, an operation reception unit 106, a blood pressure calculation condition determination unit 107, and an arrhythmia detection condition determination unit 108. These functional modules are implemented by reading a program stored in the storage unit 40. These functional modules will be described in detail later.

The pulse wave information acquisition unit 200 includes a cuff 210, a pressure sensor 221, an analog front end (AFE) 222 including an analog-to-digital (A/D) converter, a pressurizing pump 231, a pump drive circuit 232, an air release valve 241, and a valve drive circuit 242. The pulse wave information acquisition unit 200 is a functional unit that acquires information of the pressure pulse wave of the user with the above configuration, and the blood pressure of the user is measured by a so-called oscillometric method on the basis of the acquired pulse wave information. The blood pressure measurement by the oscillometric method is a well-known technology, and thus a detailed description thereof will be omitted.

The cuff 210 is a member used with being wound around the upper arm of the user and includes the pressurizing pump 231 (pump drive circuit 232) disposed in the main body housing, an air bladder (not illustrated) communicating with the air release valve 241 (valve drive circuit 242) via an air tube (not illustrated), a belt (not illustrated) incorporating the air bladder, and the like.

Note that the belt of the cuff 210 is provided with a fixing unit (for example, a hook-and-loop fastener) for fixing the cuff 210 to the upper arm of the user, and the cuff 210 is wound around the upper arm of the user by the belt when blood pressure measurement is performed using the blood pressure measurement device 1.

For example, a piezoresistive sensor including a piezoelectric element can be adopted as the pressure sensor 221, and a pressure pulse wave of the user is acquired. Furthermore, the blood pressure measurement device 1 according to the present embodiment calculates the blood pressure and the pulse of the user on the basis of the pressure pulse wave acquired by the pressure sensor 221, and detects the body motion and arrhythmia of the user during the blood pressure measurement as described later.

At the time of blood pressure measurement, the control unit 100 controls the pressurizing pump 231 (pump drive circuit 232) and the air release valve 241 (valve drive circuit 242) to adjust the cuff pressure of the cuff 210. Specifically, control is performed such that the pressurizing pump 231 is driven to send air to the cuff 210 to inflate the cuff 210 (increase the cuff pressure). In this way, after the blood flow is once inhibited by compressing the blood vessel of the upper arm of the user, control is performed such that the pressurizing pump 231 is stopped to open the air release valve 241 and the air is gradually released from the cuff 210 to contract the cuff 210 (reduce the cuff pressure).

Next, functional modules illustrated in FIG. 2 will be described. The blood pressure value calculation unit 101 calculates the blood pressure value of the user on the basis of the pressure pulse wave acquired by the pressure sensor 221. The pulse calculation unit 102 calculates the pulse rate of the user on the basis of the pressure pulse wave (such as the peak number of the pulse wave) acquired by the pressure sensor 221 and determines a pulse rate level. The pulse rate level can be decided by a plurality of (for example, three) threshold values set stepwise using the pulse rate as an index.

The body motion determination unit 103 detects the body motion of the user during acquisition of the pulse wave information (hereinafter, sometimes referred to as during blood pressure measurement) and determines a body motion level on the basis of the pressure pulse wave (such as disturbance of the pulse waveform) acquired by the pressure sensor 221. The body motion level can be decided by a plurality of (for example, four) threshold values, which are set stepwise using, as an index, the ratio of a body motion detection section (the sum of the body motion detection times in a pulse wave information acquisition period) in a section in which an envelope plotted with, for example, the cuff pressure on the horizontal axis and an amplitude of the pressure pulse wave on the vertical axis substantially forms a mountain.

Furthermore, the arrhythmia determination unit 104 determines (detects) the presence or absence of arrhythmia (for example, AF) during the blood pressure measurement on the basis of information on the pressure pulse wave (for example, information on the time interval between the peaks of the pulse wave, and the like) acquired by the pressure sensor 221. However, as described later, the determination of the presence or absence of arrhythmia by the arrhythmia determination unit 104 is executed only in a case where the biological information of the user during the acquisition of the pulse wave information satisfies a predetermined condition.

The display control unit 105 controls the display unit 20 (the display contents thereof). For example, the display control unit 105 causes the display unit 20 to display menu selection, measurement results, past history data, and the like, and specific display contents will be described later.

The operation reception unit 106 receives an operation input by a user via the operation unit 30. For example, when the user presses a measurement start button, an item selection button, a decision button, or the like (each including an interface expressed on an image), the blood pressure measurement device 1 is controlled to perform the associated operation.

The blood pressure calculation condition determination unit 107 decides whether or not to perform the blood pressure calculation by the blood pressure value calculation unit 101 (whether or not the pulse rate level and the body motion level satisfy a blood pressure calculation condition) on the basis of the pulse rate level determined by the pulse calculation unit 102 and the body motion level determined by the body motion determination unit 103.

The arrhythmia detection condition determination unit 108 decides whether or not to perform determination of presence or absence of arrhythmia by the arrhythmia determination unit 104 (whether or not the pulse rate level and the body motion level satisfy an arrhythmia detection condition) on the basis of the pulse rate level determined by the pulse calculation unit 102 and the body motion level determined by the body motion determination unit 103. That is, in the present embodiment, the pulse rate level and the body motion level correspond to the “index” according to the present invention.

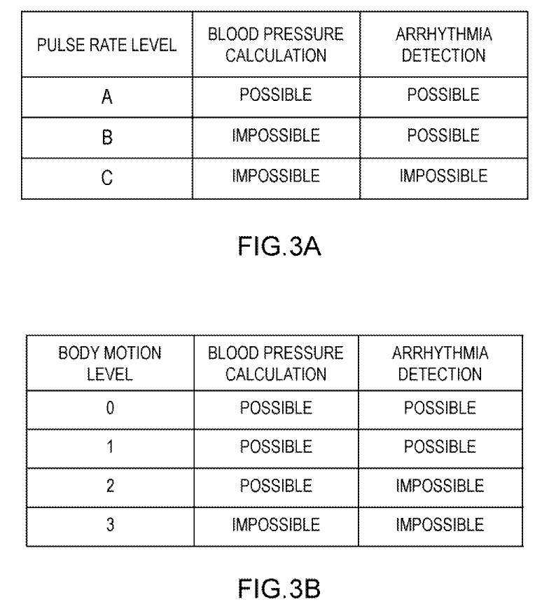

FIG. 3A is a table illustrating correspondence relationships between the pulse rate levels and possibility or impossibility of blood pressure calculation and possibility or impossibility of arrhythmia detection. FIG. 3B is a table illustrating correspondence relationships between the body motion levels and possibility or impossibility of blood pressure calculation and possibility or impossibility of arrhythmia detection. As can be seen with reference to FIGS. 3A and 3B, in a case where the pulse rate level is A and the body motion level is 2 or less, the blood pressure calculation condition determination unit 107 determines that the blood pressure calculation is possible (the blood pressure calculation condition is satisfied) with the acquired pulse wave information, and determines that the blood pressure calculation is impossible when the above condition is not satisfied. In a case where the pulse rate level is A or C and the body motion level is 1 or less, the arrhythmia detection condition determination unit 108 determines that the detection of arrhythmia is possible (the arrhythmia detection condition is satisfied) with the acquired pulse wave information, and determines that the detection of arrhythmia (determination of the presence or absence of arrhythmia) is impossible when the above condition is not satisfied.

### Display Screen Example for Each Condition

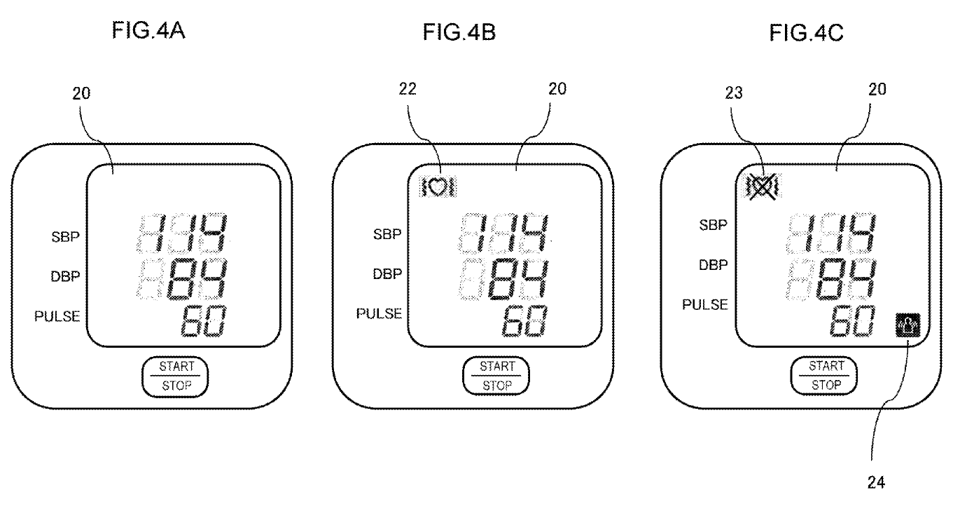

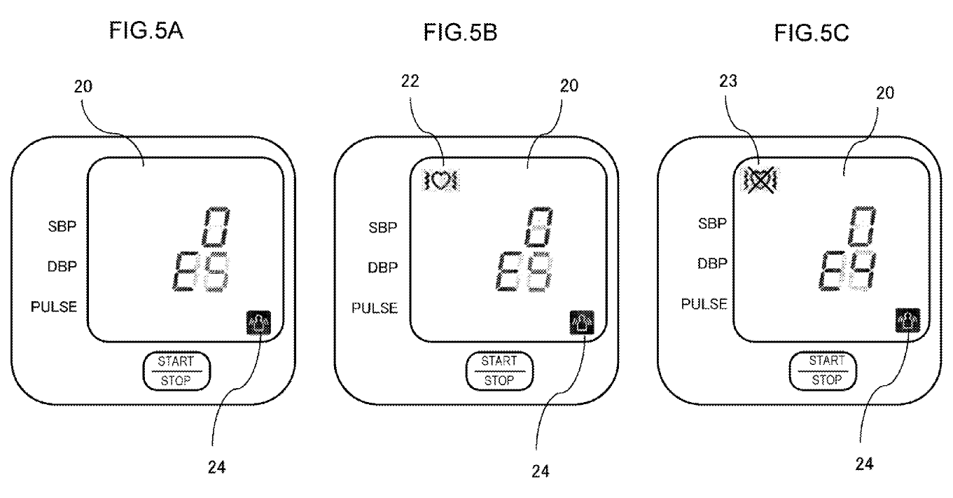

Next, with reference to FIGS. 4A to 4C and 5A to 5C, examples of a screen displayed on the display unit 20 as a result of blood pressure measurement will be described. FIGS. 4A to 4C and 5A to 5C are diagrams illustrating the display unit 20 (and its periphery) of the blood pressure measurement device 1.

FIG. 4A is a diagram illustrating an example of display of a measurement result screen in a case where the blood pressure value has been calculated and determination of the presence or absence of arrhythmia has also been performed, and where no arrhythmia has been detected. FIG. 4B is a diagram illustrating an example of display of the measurement result screen in a case where the blood pressure value has been calculated and determination of the presence or absence of arrhythmia has also been performed, and where arrhythmia has been detected. FIG. 4C is a diagram illustrating an example of display of the measurement result screen in a case where the blood pressure value has been calculated but determination of the presence or absence of arrhythmia has not been performed.

In a case where the blood pressure calculation condition has been satisfied and the arrhythmia detection condition has also been satisfied, and where no arrhythmia has been detected, as illustrated in FIG. 4A, the display unit 20 displays only the blood pressure value and the pulse rate.

In a case where the blood pressure calculation condition has been satisfied and the arrhythmia detection condition has also been satisfied, and where arrhythmia has been detected, as illustrated in FIG. 4B, the display unit 20 displays information (an arrhythmia detected icon) 22 of a fact that arrhythmia has been detected during the blood pressure measurement in addition to the blood pressure value and the pulse rate.

In a case where the blood pressure calculation condition has been satisfied but the arrhythmia detection condition has not been satisfied, as illustrated in FIG. 4C, the display unit 20 displays information (an arrhythmia detection function stopped icon) 23 of a fact that the determination of the presence or absence of arrhythmia has not been performed in addition to the blood pressure value and the pulse rate. Furthermore, in this case, information (a body motion icon) 24 indicating the cause for which the determination of the presence or absence of arrhythmia has not been performed is also displayed.

FIG. 5A is a diagram illustrating an example of display of the measurement result screen in a case where the blood pressure value has not been calculated and determination of the presence or absence of arrhythmia has been performed, and where no arrhythmia has been detected. FIG. 5B is a diagram illustrating an example of display of the measurement result screen in a case where the blood pressure value has not been calculated and determination of the presence or absence of arrhythmia has been performed, and where arrhythmia has been detected. FIG. 5C is a diagram illustrating an example of display of the measurement result screen in a case where the blood pressure value has not been calculated and determination of the presence or absence of arrhythmia has also not been performed.

In a case where the blood pressure calculation condition has not been satisfied and the arrhythmia detection condition has been satisfied, and where no arrhythmia has been detected, as illustrated in FIG. 5A, the display unit 20 displays an error code in a field for displaying the blood pressure value and the pulse rate. In addition, information (a body motion icon) 24 indicating the cause for which the blood pressure calculation could not be performed is also displayed.

In a case where the blood pressure calculation condition has not been satisfied and the arrhythmia detection condition has been satisfied, and where arrhythmia has been detected, as illustrated in FIG. 5B, the display unit 20 displays information (the arrhythmia detected icon) 22 of a fact that arrhythmia has been detected during the blood pressure measurement in addition to the error code and the body motion icon 24.

In a case where the blood pressure calculation condition has not been satisfied and the arrhythmia detection condition has also not been satisfied, as illustrated in FIG. 5C, the display unit 20 displays information (the arrhythmia detection function stopped icon) 23 of a fact that the determination of the presence or absence of arrhythmia has not been performed in addition to the error code and the body motion icon 24.

### Flow of Blood Pressure Measurement Processing

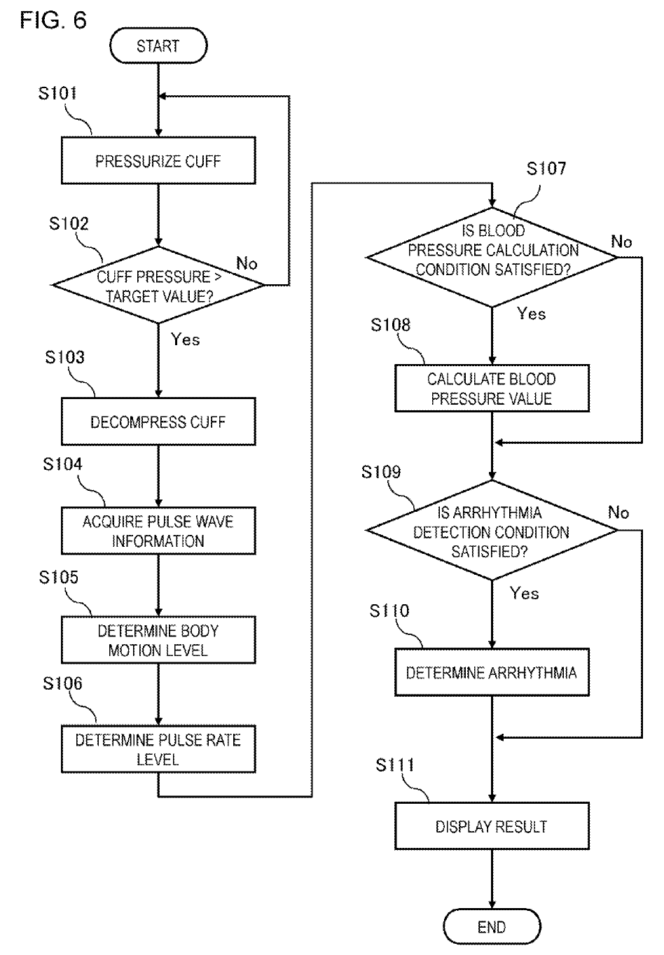

The blood pressure measurement device 1 has a mode of measuring the blood pressure of the user and displaying the measurement result on the display unit 20 (normal mode). Next, a flow of processing when measurement of blood pressure is executed by the normal mode will be described on the basis of FIG. 6. FIG. 6 is a flowchart illustrating a flow of processing by the blood pressure measurement device 1 when measurement of the blood pressure is performed. Prior to the execution of the blood pressure measurement, the user winds the cuff 210 around his/her upper arm, and then instructs execution of the blood pressure measurement in the normal mode via the operation unit 30.

After receiving the instruction of the blood pressure measurement, the blood pressure measurement device 1 (pulse wave information acquisition unit 200) drives the pressurizing pump 231 to pressurize the site where the cuff 210 is worn (S101). Then, the pulse wave information acquisition unit 200 determines whether or not the cuff pressure has reached the predetermined target value (S102), and in a case where it has been determined that the cuff pressure has not reached the predetermined cuff pressure, the process returns to step S101 to repeat the processing. On the other hand, in a case where it has been determined in step S102 that the cuff pressure has reached the predetermined target value, the pulse wave information acquisition unit 200 opens the air release valve 241 to decompress the cuff 210 (S103), and acquires information on the pressure pulse wave from the signal of the pressure sensor 221 in the process of decompression (S104).

Next, based on the information acquired in step S104, the body motion determination unit 103 performs determination of the body motion level (S105). Furthermore, the pulse calculation unit 102 performs determination of the pulse rate level (S106). Since the body motion level determination and the pulse rate level determination are as described above, the description thereof will not be repeated.

Subsequently, the blood pressure calculation condition determination unit 107 determines whether or not the blood pressure calculation condition is satisfied on the basis of the body motion level and the pulse rate level acquired in steps S105 and S106, respectively (S107). Since determination of the blood pressure calculation condition is as described above, the description thereof will not be repeated. Here, in a case where the blood pressure calculation condition determination unit 107 has determined that the blood pressure calculation condition is satisfied, the blood pressure value calculation unit 101 calculates the blood pressure value (S108) and performs the processing of step S109. On the other hand, in a case where the blood pressure calculation condition determination unit 107 has determined in step S107 that the blood pressure calculation condition is not satisfied, the processing of step S108 is skipped, and the process proceeds to step S109.

In step S109, the arrhythmia detection condition determination unit 108 determines whether or not the arrhythmia detection condition is satisfied on the basis of the body motion level and the pulse rate level acquired in steps S105 and S106, respectively (S109). Here, in a case where the arrhythmia detection condition determination unit 108 has determined that the arrhythmia detection condition is satisfied, the arrhythmia determination unit 104 performs determination of the presence or absence of arrhythmia during the acquisition of the pulse wave information (S110). On the other hand, in a case where the arrhythmia detection condition determination unit 108 has determined in step S109 that the arrhythmia detection condition is not satisfied, the processing of step S110 is skipped, and the process proceeds to step S111.

In step S111, the display control unit 105 displays (S111) the measurement result on the display unit 20 according to the patterns of whether or not the blood pressure calculation has been performed, whether or not the determination of the presence or absence of arrhythmia has been performed, and, in a case where the determination of the presence or absence of arrhythmia has been performed, whether or not arrhythmia has been detected, and ends the series of processing. Since the display screen example for each pattern has already been described with reference to FIGS. 4A to 4C and 5A to 5C, the description thereof is omitted here. Note that each piece of information such as the information on the pressure pulse wave acquired by the pulse wave information acquisition unit 200, the blood pressure value and the pulse rate calculated by the blood pressure value calculation unit 101, the pulse rate level decided by the pulse calculation unit 102, the body motion level decided by the body motion determination unit 103, the presence or absence of arrhythmia determined by the arrhythmia determination unit 104, the determination result by the blood pressure calculation condition determination unit 107, and the determination result by the arrhythmia detection condition determination unit 108 is stored in the storage unit 40 at the end of processing or at an appropriate timing during processing.

### Flow of Past Data Display Processing

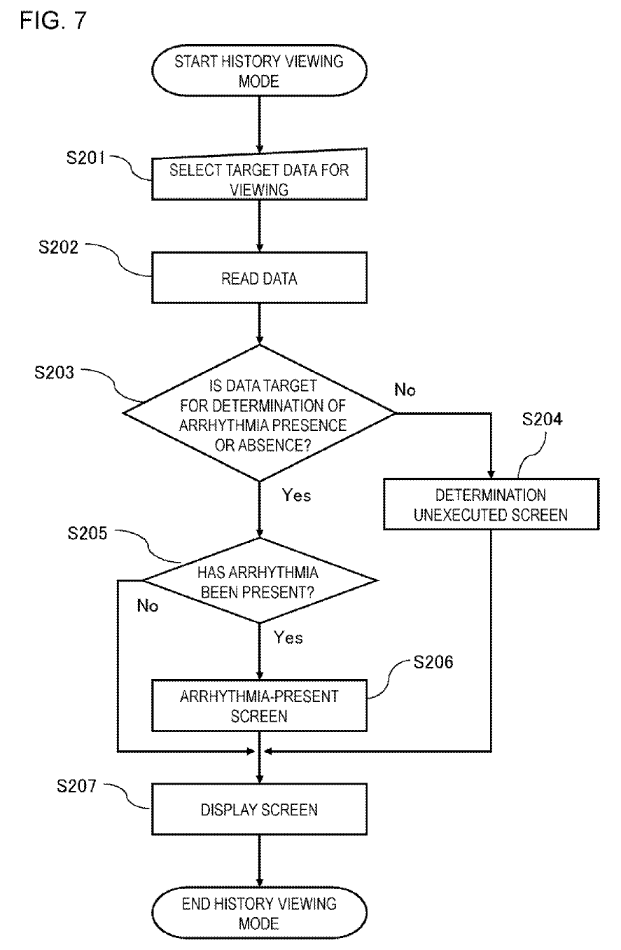

The blood pressure measurement device 1 has a mode in which past measurement data stored in the storage unit 40 is displayed on the display unit 20 and viewed (history viewing mode). Here, a flow of processing when past measurement data is displayed on the display unit 20 by the history viewing mode will be described on the basis of FIG. 7.

FIG. 7 is a flowchart illustrating a flow of processing of referring to past measurement data (history viewing mode) in the blood pressure measurement device 1. As illustrated in FIG. 7, first, past measurement data (for example, which is read from the storage unit 40 and displayed as a list on the display unit 20) that the user wants to refer to is selected via the operation unit 30 (S201), and the blood pressure measurement device 1 reads the measurement data related to the selection by the user from the storage unit 40 (S202).

Next, the blood pressure measurement device 1 determines whether or not the data read in step S202 has been a target for determination of the presence or absence of arrhythmia (that is, whether or not the arrhythmia detection condition has been satisfied) (S203). Here, in a case where it has been determined that the data has not been the target for determination of the presence or absence of arrhythmia, a screen indicating information of a fact that the determination of the presence or absence of arrhythmia has not been performed is displayed on the display unit 20 together with the measurement value (S204, S207).

In a case where it has been determined in step S203 that the data related to the viewing request has been a target for determination of the presence or absence of arrhythmia, the blood pressure measurement device 1 determines whether or not the data is data of a case where arrhythmia has been detected during blood pressure measurement (S205). Here, in a case where it has been determined that the data is data of a case where arrhythmia has been detected, a screen indicating information of a fact that arrhythmia has been detected during the pulse wave information acquisition is displayed on the display unit 20 together with the measurement value (S206, S207). On the other hand, in a case where it has been determined in step S205 that no arrhythmia has been detected, only the measurement value is displayed on the display unit 20 (S207). After displaying the data related to the viewing request on the display unit 20 in step S207, the blood pressure measurement device 1 ends the processing of the history viewing mode.

According to the blood pressure measurement device 1 as described above, it is possible to display different measurement result screens according to the patterns of whether or not the blood pressure calculation has been performed, whether or not the determination of the presence or absence of arrhythmia has been performed, and, in a case where the determination of the presence or absence of arrhythmia has been performed, whether or not arrhythmia has been detected. As a result, it is possible to prevent the user from erroneously recognizing that “arrhythmia has been absent” during the blood pressure measurement even though determination of the presence or absence of arrhythmia has merely not been performed during the blood pressure measurement (that is, the arrhythmia detection function has been canceled). Furthermore, in a case where the determination of the presence or absence of arrhythmia has not been performed, by also indicating the information indicating the cause for that, the user can more clearly recognize that the determination of the presence or absence of arrhythmia has not been performed.

### Modification

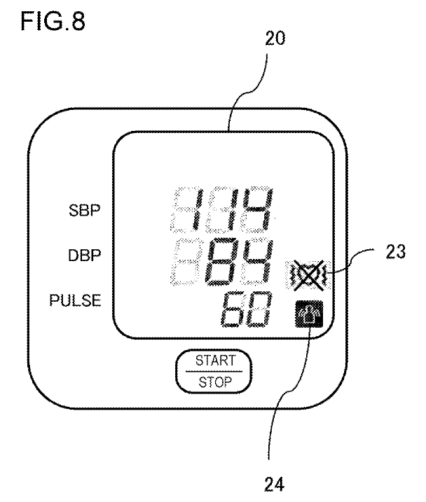

Note that the measurement result screen illustrated in the above embodiment is an example, and the display mode of the blood pressure measurement result can be appropriately changed. FIG. 8 illustrates a screen example according to such a modification. As illustrated in FIG. 8, in the display screen example according to the present modification, the arrhythmia detection function stopped icon 23 indicating a fact that the determination of the presence or absence of arrhythmia during the blood pressure measurement has not been performed and the body motion icon 24 indicating the cause therefor are displayed at positions close to each other. By aggregating the related information in one place in this manner, listing visibility can be improved, and the user can more easily grasp the information.

In the above example, the stop of the arrhythmia detection function, the cause therefor (body motion), and the information of the presence of arrhythmia in the case where determination of the presence or absence of arrhythmia has been performed are displayed as icons on the display unit 20, but these pieces of information may be displayed by other methods. For example, such a display method may be adopted in which words such as “arrhythmia detection function stopped”, “arrhythmia present”, and “body motion present” are described on name plates around the display unit 20, and in the case corresponding to each description content, a symbol or a figure is displayed on the display unit 20 at a position adjacent to the description on the name plate to indicate the description of the words.

In addition, such a display may be adopted in which, instead of simultaneously displaying the measurement values (blood pressure value, pulse rate) and the icons, the measurement values and the icons are alternately displayed.

### Second Embodiment

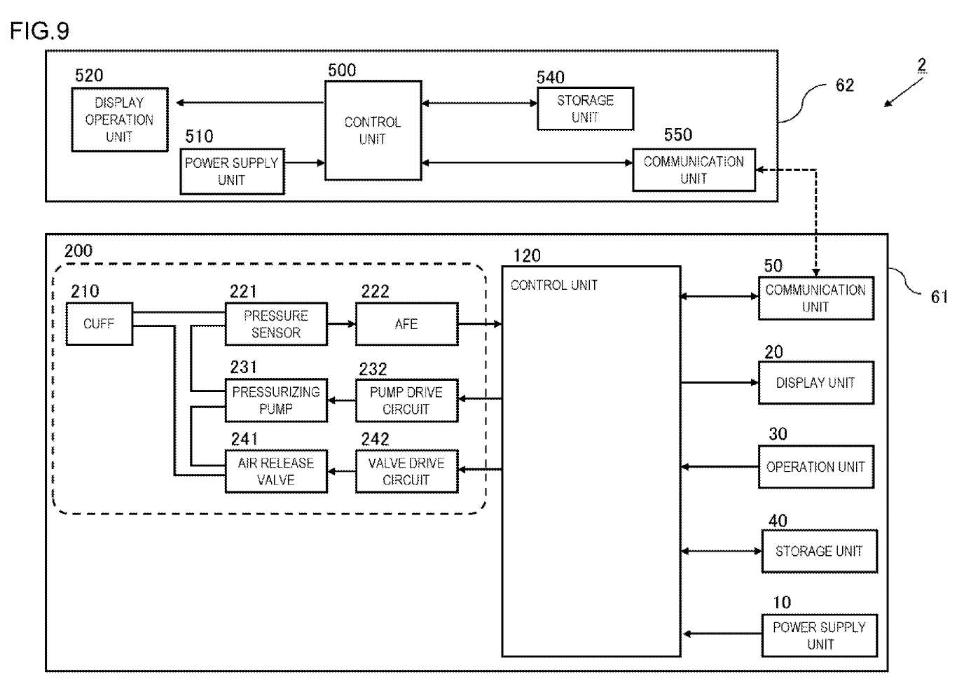

Next, another embodiment will be described on the basis of FIGS. 9 to 13. In each of the above embodiments, the detection of arrhythmia during the blood pressure measurement is executed in the blood pressure measurement device, but the detection of arrhythmia during the blood pressure measurement may be executed in another device. Hereinafter, an embodiment as a system including the blood pressure measurement device and the information processing terminal will be described.

FIG. 9 is a block diagram schematically illustrating a configuration of a biological information processing system 2 according to the present embodiment. As illustrated in FIG. 9, the biological information processing system 2 according to the present embodiment includes a blood pressure measurement device 61 including the pulse wave information acquisition unit 200, and a smartphone 62 as an example of the information processing terminal, which are configured to be communicably connectable.

The blood pressure measurement device 61 has a hardware configuration substantially similar to that of the blood pressure measurement device 1 according to the first embodiment, and is only different in including a communication unit 50. Hereinafter, the same reference numerals will be used for the same configurations as those already described, and repeated description will be omitted.

The communication unit 50 functions to communicate with and connect to the smartphone 62, and can adopt an appropriate communication interface according to a communication network to be connected, such as a wireless communication antenna. Note that a communication method between the blood pressure measurement device 61 and the smartphone 62 is not particularly limited, and for example, pairing by Bluetooth (registered trademark) low energy (BLE) communication, communication by Wi-Fi (registered trademark) (LAN), or the like can be adopted.

As illustrated in FIG. 9, the smartphone 62 includes configurations such as a power supply unit 510, a display operation unit 520, a storage unit 540, a communication unit 550, and a control unit 500.

The power supply unit 510 is a power source of the smartphone 62, and can be, for example, a rechargeable secondary battery such as a lithium-ion battery. The display operation unit 520 can be a touch panel display or the like having both functions of information display and input operation reception.

The storage unit 540 includes a main storage device such as a RAM and a ROM, and an auxiliary storage device such as a flash memory, and stores various types of information such as an application program and various pieces of biological information. Note that the measurement results of the blood pressure and the like and other acquired biological information may be stored in the storage unit 540 in association with time information such as the measurement (acquisition) time. As the time information, for example, time information clocked by referring to an RTC of the control unit 500 can be used. Furthermore, the auxiliary storage device may be configured to be detachable from the main body housing (not illustrated).

The communication unit 550 functions to communicate with and connect to the blood pressure measurement device 61, and can adopt an appropriate communication interface according to a communication network to be connected, such as a wired communication port or a wireless communication antenna.

The control unit 500 is a functional unit that controls the smartphone, and includes, for example, a processor such as a CPU and a GPU. When receiving a user's operation via the display operation unit 520, the control unit 500 controls each component of the smartphone to exhibit various functions according to a predetermined application program. Note that the predetermined program is stored in the storage unit 540 and read therefrom.

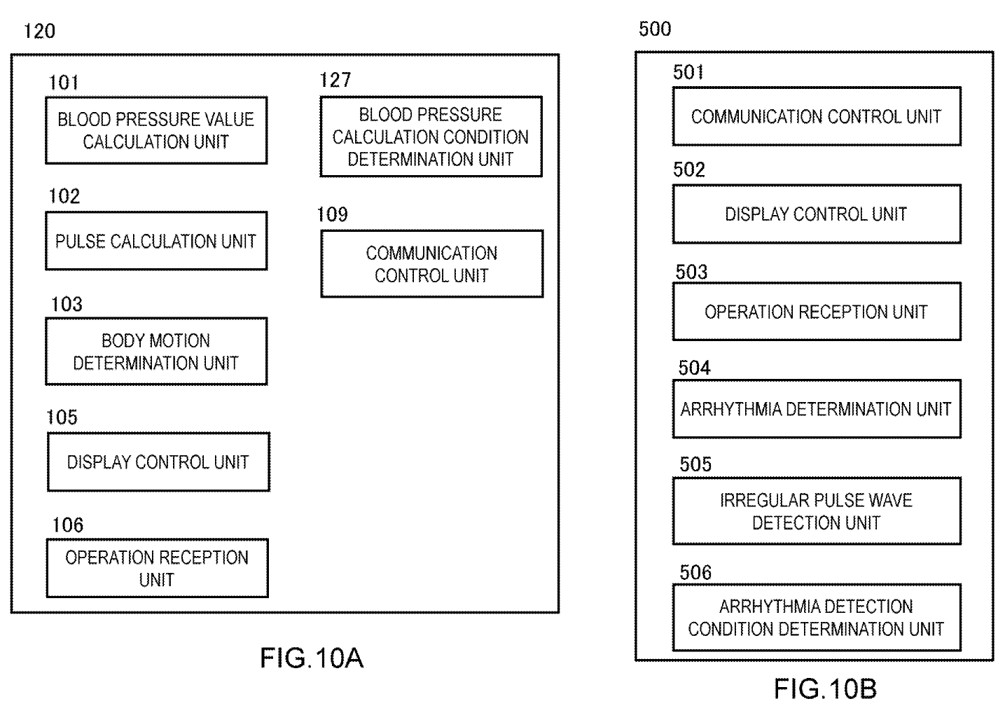

FIG. 10A is a block diagram illustrating part of functional modules implemented by the control unit 120 of the blood pressure measurement device 61, and FIG. 10B is a block diagram illustrating part of functional modules implemented by the control unit 500 of the smartphone 62.

As illustrated in FIG. 10A, the functional modules of the blood pressure measurement device 61 are different from those of the blood pressure measurement device 1 of the first embodiment in that a communication control unit 109 is added and the arrhythmia determination unit does not exist. Unlike the blood pressure calculation condition determination unit 127 of the first embodiment, the blood pressure calculation condition determination unit 127 determines whether or not the blood pressure calculation is possible only with the body motion level. The communication control unit 109 controls communication with an external device (for example, the smartphone 62) by the communication unit 50.

As illustrated in FIG. 10B, the control unit 500 of the smartphone 62 implements functional modules including a communication control unit 501, a display control unit 502, an operation reception unit 503, an arrhythmia determination unit 504, an irregular pulse wave detection unit 505, and an arrhythmia detection condition determination unit 506. The communication control unit 501 controls communication with an external device (for example, the blood pressure measurement device 61) by the communication unit 550.

The display control unit 502 controls the display contents of the display operation unit 520. For example, by executing an application program related to cooperation with the blood pressure measurement device 61, the display control unit 502 causes the display operation unit 520 to display information related to determination of the presence or absence of arrhythmia by the arrhythmia determination unit 504 described later. In addition to this, the display control unit 502 causes the display operation unit 520 to display a menu screen (operation reception screen), guidance related to the progress of biological information measurement, a measurement result of biological information, and the like.

The operation reception unit 503 receives an input of a user operation via a user interface displayed on the display operation unit 520. For example, by execution of an application program related to cooperation with the blood pressure measurement device 61, the operation reception unit 503 receives an operation input such as selection of an item and screen transition.

As will be described later, the arrhythmia determination unit 504 determines presence or absence of arrhythmia of the user during blood pressure measurement on the basis of information transmitted from the blood pressure measurement device 61 (including pulse wave information). That is, in the present embodiment, the detection processing of arrhythmia during the blood pressure measurement is performed not by the blood pressure measurement device 61 but by the smartphone 62. However, as described later, the determination of the presence or absence of arrhythmia by the arrhythmia determination unit 504 is executed only in a case where the biological information of the user during the acquisition of the pulse wave information satisfies a predetermined condition.

The irregular pulse wave detection unit 505 determines (detects) the presence or absence and the number of irregular pulse waves in the pulse wave of the user during blood pressure measurement on the basis of the pulse wave information transmitted from the blood pressure measurement device 61. Specifically, for example, an average value of pulse wave intervals (widths between peaks) is obtained from the pulse wave information (waveform data), and when there is a pulse wave interval deviated from the calculated average value by a predetermined ratio (for example, ±25%) or more, a waveform portion related to the deviated pulse wave interval is determined as an irregular pulse wave.

Based on the information on the body motion level transmitted from the blood pressure measurement device 61 and the number of irregular pulse waves determined by the irregular pulse wave detection unit 505, the arrhythmia detection condition determination unit 506 determines whether or not to perform determination of the presence or absence of arrhythmia by the arrhythmia determination unit 504 (whether or not the body motion level and the number of irregular pulse waves satisfy the arrhythmia detection condition). That is, in the present embodiment, the body motion level and the number of irregular pulse waves correspond to the “index” according to the present invention.

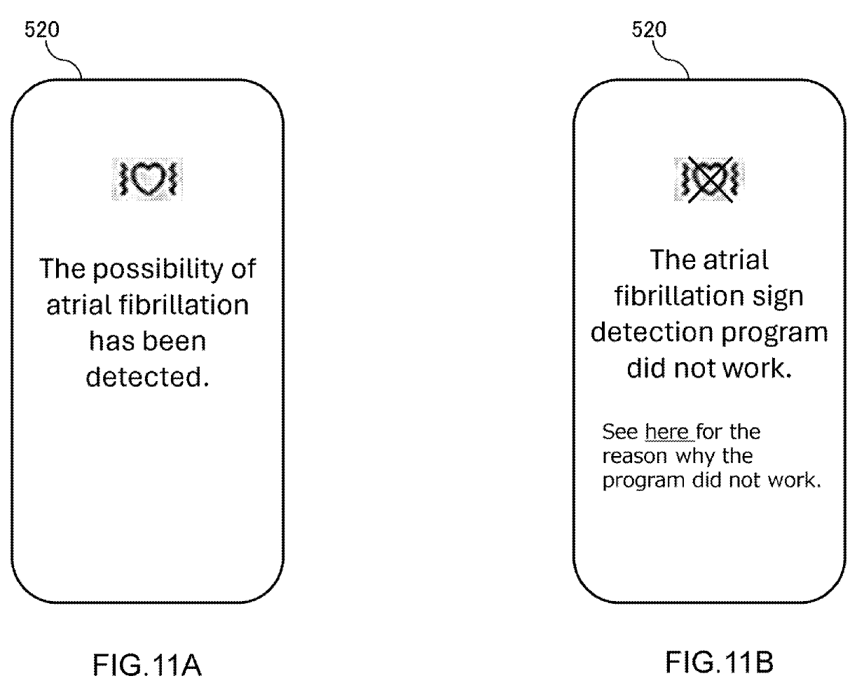

In a case where the arrhythmia detection condition determination unit 506 has determined that the body motion level and the number of irregular pulse waves during the blood pressure measurement satisfy the arrhythmia detection condition and then the arrhythmia determination unit 504 has determined that arrhythmia (atrial fibrillation) has been present during the blood pressure measurement, a screen as illustrated in FIG. 11A is displayed on the display operation unit 520. In a case where the arrhythmia detection condition determination unit 506 has determined that the body motion level and the number of irregular pulse waves during the blood pressure measurement do not satisfy the arrhythmia detection condition, a screen as illustrated in FIG. 11B is displayed on the display operation unit 520.

### Flow of Processing

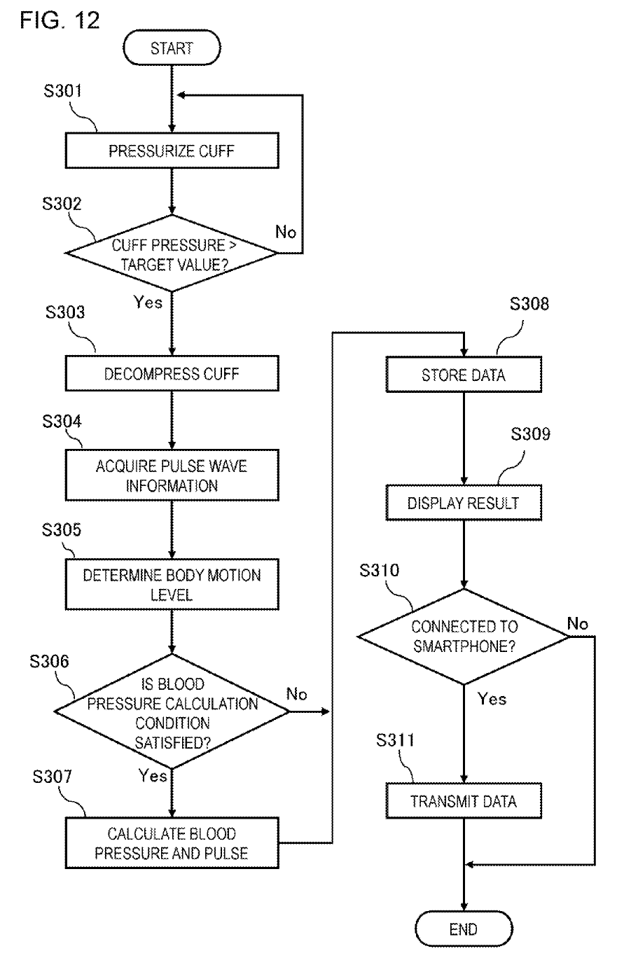

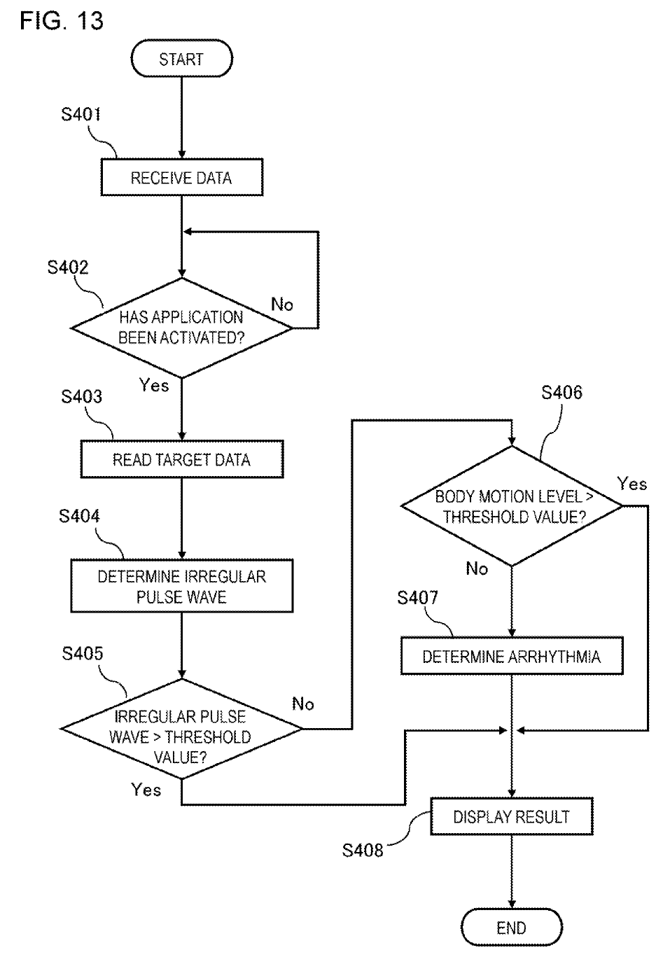

Subsequently, based on FIGS. 12 and 13, flows of blood pressure measurement processing, determination processing of presence or absence of arrhythmia, and measurement result display processing executed in the biological information processing system 2 according to the present embodiment will be described. FIG. 12 is a flowchart illustrating a flow of an example of processing executed by the blood pressure measurement device 61 of the biological information processing system 2 according to the present embodiment. FIG. 13 is a diagram illustrating a flow of an example of processing executed by the smartphone 62 of the biological information processing system 2.

Prior to the execution of the blood pressure measurement by the blood pressure measurement device 61, the user winds the cuff 210 around his/her upper arm and then instructs execution of the blood pressure measurement via the operation unit 30. After receiving the instruction of the blood pressure measurement, the blood pressure measurement device 61 (pulse wave information acquisition unit 200) drives the pressurizing pump 231 to pressurize the site where the cuff 210 is worn (S301). Then, the pulse wave information acquisition unit 200 determines whether or not the cuff pressure has reached the predetermined target value (S302), and in a case where it has been determined that the cuff pressure has not reached the predetermined cuff pressure, the process returns to step S301 to repeat the processing. On the other hand, in a case where it has been determined in step S302 that the cuff pressure has reached the predetermined target value, the pulse wave information acquisition unit 200 opens the air release valve 241 to decompress the cuff 210 (S303) and acquires information on the pressure pulse wave from the signal of the pressure sensor 221 in the process of decompression (S304).

Next, based on the information acquired in step S304, the body motion determination unit 103 performs determination of the body motion level (S305). Thereafter, based on the body motion level determined in step S307, the blood pressure calculation condition determination unit 127 determines whether or not calculation of the blood pressure value is possible on the basis of the acquired pulse wave information (S306). Here, in a case where the blood pressure calculation condition determination unit 127 has determined that calculation of the blood pressure value is possible, the blood pressure value calculation unit 101 and the pulse calculation unit 102 perform calculation of the blood pressure value and the pulse rate, respectively (S307). Then, various kinds of biological information such as the blood pressure value and the pulse rate calculated in step S307, the pulse wave information acquired in step S304, and the body motion level determined in step S305 are stored in the storage unit 40 (S308).

On the other hand, in a case where the blood pressure calculation condition determination unit 127 has determined in step S306 that the blood pressure calculation condition is not satisfied, the processing of step S307 is skipped, and the process transitions to processing of step S308. In this case, as a matter of course, the data stored in step S308 does not include the blood pressure value and the pulse rate.

After the data is stored in step S308, the display control unit 105 displays the measurement result on the display unit 20 (S309). In a case where it has been determined in step S306 that the blood pressure calculation condition is not satisfied, what is displayed on the display unit 20 in step S309 is the error code (and the body motion icon).

Next, the communication control unit 109 performs determination of whether or not a connection with the smartphone 62 has been established (S310). Here, in a case where it has been determined that the communication connection with the smartphone 62 has been established, the communication control unit 109 transmits at least the pulse wave information acquired in step S304 and the information including the body motion level determined in step S305 (hereinafter, also referred to as measurement data) to the smartphone 62 (S311), and ends the series of processing. On the other hand, in a case where it has been determined in step S310 that the communication connection with the smartphone 62 has not been established, the processing of step S311 is skipped, and the series of processing ends.

Next, a flow of processing performed by the smartphone 62 will be described with reference to FIG. 13. In the following description, description will be made assuming that a communication connection between the smartphone 62 and the blood pressure measurement device 61 is in an established state. As illustrated in FIG. 13, first, in the smartphone 62, the communication control unit 501 receives the measurement data from the blood pressure measurement device 61 via the communication unit 50 (S401).

Next, the control unit 500 determines whether or not the application program related to the arrhythmia detection (hereinafter, simply referred to as application) has been activated (S402), and repeats the processing of step S402 (waits for the application activation by the user) in a case where it has been determined that the application program has not been activated. On the other hand, in a case where it has been determined in step S402 that the application has been activated, the control unit 500 (application) reads measurement data to be a target for arrhythmia detection (for example, measurement data at the time of most recent blood pressure measurement) (S403).

Next, the irregular pulse wave detection unit 505 determines the presence or absence and the number of irregular pulse waves of the user during blood pressure measurement on the basis of the pulse wave information in measurement data read in step S403 (S404). Since specific processing is as described above, the description thereof will not be repeated. Next, the arrhythmia detection condition determination unit 506 determines whether the number of irregular pulse waves detected in step S404 exceeds a predetermined threshold value (S405). Here, in a case where it has been determined that the number of irregular pulse waves exceeds the predetermined threshold value (that is, in a case where it has been determined that the arrhythmia detection condition is not satisfied), the display control unit 502 causes the display operation unit 520 to display a screen (the screen illustrated in FIG. 11B) of a fact that the determination of the presence or absence of arrhythmia has not been performed (S408), and ends the series of processing.

On the other hand, in a case where it has been determined in step S405 that the number of irregular pulse waves is equal to or less than the threshold value, the arrhythmia detection condition determination unit 506 determines whether or not the body motion level included in the measurement data exceeds the threshold value (S406). Here, in a case where it has been determined that the body motion level exceeds the predetermined threshold value (that is, in a case where it has been determined that the arrhythmia detection condition is not satisfied), the display control unit 502 causes the display operation unit 520 to display a screen indicating a fact that the determination of the presence or absence of arrhythmia has not been performed (S408), and ends the series of processing.

In a case where it has been determined in step S406 that the body motion level is equal to or less than the predetermined threshold value (that is, in a case where it has been determined that the arrhythmia detection condition is satisfied), the arrhythmia determination unit 504 performs determination of presence or absence of arrhythmia during the acquisition of the pulse wave information related to the target data (S407). Then, in a case where the arrhythmia determination unit 504 has determined that arrhythmia has been present during the acquisition of the pulse wave information, the display control unit 502 causes the display operation unit 520 to display a screen (a screen illustrated in FIG. 11A) notifying that arrhythmia has been present during the acquisition of the pulse wave information (S408), and ends the series of processing. Note that, in a case where the arrhythmia determination unit 504 has determined in step S407 that arrhythmia has been absent during the acquisition of the pulse wave information, the series of processing is ended without performing special display on the display operation unit 520.

According to the biological information processing system 2 as described above, the determination of the presence or absence of arrhythmia during the blood pressure measurement can be executed by the information processing terminal such as the smartphone 62 instead of the blood pressure measurement device 61. As a result, the calculation load of the blood pressure measurement device can be reduced.

### Others

Note that the description of the above-described embodiments is merely illustrative of the present invention, and the present invention is not limited to the above-described specific forms. The present invention can be variously modified and combined within the scope of the technical idea. For example, in each of the above examples, the body motion level, the pulse rate, the irregular pulse wave number, and the like are exemplified as the “index” to be a target for determination of the arrhythmia detection condition, but other indexes can be used instead of or in addition to these indexes. Specifically, for example, the magnitude of the amplitude of the pulse waveform (if it is small, the condition is not satisfied) can be used as an index. In the first embodiment, the body motion level and the pulse rate are combined with each other, and in the second embodiment, the body motion level and the irregular pulse wave number are combined (two indexes are used) to perform determination of whether or not the arrhythmia detection condition is satisfied. However, such a determination can be performed using any index alone.

In the above embodiments, the body motion determination unit 103 determines the presence or absence and the number of times of the body motion during the blood pressure measurement on the basis of the pressure pulse wave (such as disturbance of the pulse waveform) acquired by the pressure sensor 221, but the body motion may be detected by other methods. For example, such a configuration may also be adopted in which a motion sensor such as an acceleration sensor is disposed in the cuff 210 and the body motion is detected on the basis of an output signal of the motion sensor.

In the above embodiment, the blood pressure value is calculated by the oscillometric method, that is, the pressure pulse wave is acquired as the pulse wave information, but the type of pulse wave and the blood pressure value calculation method are not limited thereto. For example, the blood pressure measurement device may acquire a photoelectric pulse wave and calculate blood pressure by a photoelectric pulse wave method. In each of the above examples, the description has been made using the atrial fibrillation as an example of the arrhythmia, but naturally, other arrhythmia (atrial flutter, premature contraction, etc.) is also a target. In the second embodiment described above, the smartphone 62 is exemplified as the information processing terminal, but the information processing terminal is not limited to the smartphone 62, and other computers can be adopted regardless of whether the computer is a portable computer or a stationary computer.

What is claimed is:

1. A blood pressure measurement device that measures blood pressure of a living body, the blood pressure measurement device comprising:
a pulse wave acquisition unit that acquires pulse wave information;
a display unit including an area capable of displaying at least the blood pressure measured; and
a control unit that performs control of the blood pressure measurement device, wherein
the control unit
performs determination of presence or absence of arrhythmia during acquisition of the pulse wave information in a case where one or more indexes satisfy an arrhythmia detection condition, the one or more indexes being related to determination of possibility or impossibility of detection of arrhythmia in the living body at a time of the acquisition of the pulse wave information,
causes, in a case where the one or more indexes have satisfied the arrhythmia detection condition, and where it has been determined that arrhythmia has been present during the acquisition of the pulse wave information, the display unit to display information of a fact that arrhythmia has been present during the acquisition of the pulse wave information, and
causes, in a case where the one or more indexes have not satisfied the arrhythmia detection condition, the display unit to display information of a fact that determination of presence or absence of arrhythmia during the acquisition of the pulse wave information has not been performed.

2. The blood pressure measurement device according to claim 1, wherein
the one or more indexes include a body motion level during the acquisition of the pulse wave information of the living body, and
the control unit
determines whether or not the arrhythmia detection condition is satisfied using information related to body motion of the living body at the time of the acquisition of the pulse wave information.

3. The blood pressure measurement device according to claim 1, wherein
the one or more indexes include a pulse rate level during the acquisition of the pulse wave information of the living body, and
the control unit
determines whether or not the arrhythmia detection condition is satisfied using information on a pulse rate of the living body at the time of the acquisition of the pulse wave information.

4. The blood pressure measurement device according to claim 2, wherein
in a case where the body motion level during the acquisition of the pulse wave information has been equal to or higher than a threshold value and the one or more indexes have not satisfied the arrhythmia detection condition,
the control unit
displays, at positions close to each other on the display unit, information of a fact that the determination of presence or absence of arrhythmia has not been performed and information of a fact that the body motion has been detected.

5. The blood pressure measurement device according to claim 1, further comprising
a storage unit that holds information related to measurement of the blood pressure performed in a past, the information being information related to a date and time of the measurement and information related to presence or absence of arrhythmia during the acquisition of the pulse wave information or information of a fact that determination of presence or absence of arrhythmia has not been performed, wherein
the control unit
receives a request to display, on the display unit, the information related to the measurement performed in the past of the blood pressure, the information being held in the storage unit,
causes, in a case where information of a fact that arrhythmia has been present during the acquisition of the pulse wave information is held as the information related to the measurement of the blood pressure related to the request to display, the display unit to display the information of the fact that arrhythmia has been present during the acquisition of the pulse wave information, and causes, in a case where information of a fact that determination of presence or absence of arrhythmia has not been performed during the acquisition of the pulse wave information is held, the display unit to display information of the fact that determination of presence or absence of arrhythmia has not been performed during the acquisition of the pulse wave information.

6. A biological information processing system comprising: a blood pressure measurement device that measures blood pressure of a living body; and an information processing terminal that includes a display unit and is configured to be communicable with the blood pressure measurement device, wherein
the blood pressure measurement device is configured to
acquire biological information of the living body including at least pulse wave information and
transmit the biological information to the information processing terminal, and wherein
the information processing terminal is configured to
receive the biological information,
perform determination of presence or absence of arrhythmia during acquisition of the pulse wave information in a case where one or more indexes have satisfied an arrhythmia detection condition, the one or more indexes being related to determination of possibility or impossibility of detection of arrhythmia in the living body at a time of the acquisition of the pulse wave information,
display, in a case where the one or more indexes have satisfied the arrhythmia detection condition, and where it has been determined that arrhythmia has been present during the acquisition of the pulse wave information, information of a fact that arrhythmia has been present during the acquisition of the pulse wave information on the display unit, and
display, in a case where the one or more indexes have not satisfied the arrhythmia detection condition, information of a fact that determination of presence or absence of arrhythmia during the acquisition of the pulse wave information has not been performed on the display unit.

7. The biological information processing system according to claim 6, wherein
the one or more indexes include a body motion level during the acquisition of the pulse wave information of the living body, and
the information processing terminal
determines whether or not the arrhythmia detection condition is satisfied using information related to body motion of the living body at a time of acquisition of the pulse wave information.

8. The biological information processing system according to claim 7, wherein
the one or more indexes further include frequency of irregular pulse waves during the acquisition of the pulse wave information of the living body, and
the information processing terminal
detects the irregular pulse waves of the living body at the time of the acquisition of the pulse wave information on the basis of the biological information, and
determines whether or not the arrhythmia detection condition is satisfied further using information related to the number of times of detection of the irregular pulse waves.

9. An information processing terminal comprising a communication unit, a display unit, and a control unit, and configured to be communicably connectable to a biological information measurement device that acquires biological information including at least pulse wave information, wherein
the control unit
receives the biological information from the biological information measurement device via the communication unit,
performs determination of presence or absence of arrhythmia during acquisition of the pulse wave information in a case where one or more indexes satisfy an arrhythmia detection condition, the one or more indexes being related to determination of possibility or impossibility of detection of arrhythmia in the living body at a time of the acquisition of the pulse wave information,
causes, in a case where the one or more indexes have satisfied the arrhythmia detection condition, and where it has been determined that arrhythmia has been present during the acquisition of the pulse wave information, the display unit to display information of a fact that arrhythmia has been present during the acquisition of the pulse wave information, and
causes, in a case where the one or more indexes have not satisfied the arrhythmia detection condition, the display unit to display information of a fact that determination of presence or absence of arrhythmia during the acquisition of the pulse wave information has not been performed.

10. The information processing terminal according to claim 9, wherein
the one or more indexes include a body motion level during the acquisition of the pulse wave information of the living body, and
the control unit
determines whether or not the arrhythmia detection condition is satisfied using information related to body motion of the living body at a time of acquisition of the pulse wave information.

11. A non-transitory computer-readable storage medium recording a program for causing a computer to function as the information processing terminal according to claim 9.
